(19) United States
(12) Patent Application Publication
(10) Pub. No.: US 2017/0080171 A1
(43) Pub. Date: Mar. 23, 2017

(54) AIRWAY DEVICE

(71) Applicant: Epnevma LLC, Cedar Grove, NJ (US)

(72) Inventor: Andrew John ISKANDER, Cedar Grove, NJ (US)

(21) Appl. No.: 14/862,594

(22) Filed: Sep. 23, 2015

(51) Int. Cl. A61M 16/04

(52) U.S. Cl. CPC A61M 16/0434

(57) ABSTRACT

Embodiments of an airway device are disclosed herein. In some embodiments, an airway device includes a body having a proximal end, a distal end, and an intermediate section extending between the proximal and distal ends; at least one lumen extending through the body from the proximal end to the distal end; a bulbous portion disposed at the proximal end; and a straight portion disposed proximate the distal end.

### FIELD

Embodiments of the present disclosure generally relate to an airway tube for use during surgery.

### BACKGROUND

The supraglottic airway (SGA) is above a person's vocal cords. Presently, devices and methods for addressing airway obstruction in a supine patient are a result of the potential for catastrophic morbidity and mortality associated with obstruction in this part of the aerodigestive tract. Collapse of the SGA in the supine patient has been demonstrated to occur at the area posterior to the soft palate and at the base of the tongue. Studies of airway obstruction employing lateral cephalometry, dynamic MRI, and other methods have consistently demonstrated that collapse at these sites appears to be present even in anatomically normal subjects. The cause of such an occlusion of the airway above the larynx appears to be a combination of reasons including gravity on the naturally present morphology of the SGA and loss of physiologic mechanisms meant to maintain patency throughout its length.

Various devices have been developed to safely place or fix a tube for stenting open the airway from the oral or nasal inlet to the trachea, while posing the least possible risk to the patient. It is crucial that an airway device is able to allow for management of the airway with a high level of safety while mitigating the complications and necessary expertise inherent to more invasive methods of securement, such as endotracheal intubation. As a result, with each new device there is a subsequent need for training in how to safely use the device. Problems associated with placement of currently utilized devices are often related to their effect on the structures traversed from the airway inlet to the final position within the SGA.

Oropharyngeal airways comprise a category of devices inserted through the oral inlet that are meant to prevent and alleviate obstruction at the level of the base of the tongue. While presently used devices have been shown to be effective, there is evidence of morbidity associated with the use of these devices resulting from the method of insertion and location within the SGA. Typical problems associated with placement of oropharyngeal airways include trauma, airway reactivity, and obstruction.

Trauma results from placement of the airway device as the edges come into contact with the oropharyngeal mucosa. With conventional devices, the trauma may occur due to the entire outer length of the device acting as a leading edge that can scrape the lining of the palate and oropharynx as it is advanced and rotated during placement or from the leading end of the tube contacting the mucosal surfaces as the device is advanced. Trauma also results when the patient bites on the device, which occurs because all of the compressive forces during the bite reflex are not distributed away from the single point of contact where the front incisor meets the device.

Airway reactivity occurs because conventional devices maintain a contour that, when placed appropriately, presses on the base of tongue, which can trigger a gag reflex with subsequent regurgitation and other sequelae including risk of aspiration. Obstruction often results when the conventional device is too short, thus compressing the base of the tongue and causing it to bulge out into the pharyngeal lumen, or too long, thus pushing the epiglottis over the glottic opening.

As such, the inventor has provided embodiments of an improved airway device.

### SUMMARY

Embodiments of an airway device are disclosed herein. In some embodiments, an airway device includes a body having a proximal end, a distal end, and an intermediate section extending between the proximal and distal ends; at least one lumen extending through the body from the proximal end to the distal end; a bulbous portion disposed at the proximal end; and a straight portion disposed proximate the distal end.

In some embodiments, an airway device includes a body having a proximal end, a distal end, and an intermediate section extending between the proximal and distal ends; at least one lumen extending through the body from the proximal end to the distal end; a bulbous portion disposed at the proximal end; a flange disposed at the distal end; and a straight portion disposed proximate the distal end perpendicular to the flange.

In some embodiments, an airway device includes a body having a proximal end, a distal end, and an intermediate section extending between the proximal and distal ends; at least one lumen extending through the body from the proximal end to the distal end; a bulbous portion disposed at the proximal end; a tapered section disposed at the distal end, wherein the tapered section tapers away from the distal end towards the proximal end; and a straight portion disposed proximate the distal end extending from the tapered section towards the distal end.

Other and further embodiments of the present disclosure are described below.

### BRIEF DESCRIPTION OF THE DRAWINGS

Embodiments of the present disclosure, briefly summarized above and discussed in greater detail below, can be understood by reference to the illustrative embodiments of the disclosure depicted in the appended drawings. It is to be noted, however, that the appended drawings illustrate only typical embodiments of this disclosure and are therefore not to be considered limiting of its scope, for the disclosure may admit to other equally effective embodiments.

FIG. 1 depicts a schematic view of an airway device in accordance with some embodiments of the present disclosure.

FIG. 2 depicts a cross sectional area taken alone line 2-2′ in FIG. 1.

FIG. 3 depicts a cross sectional area taken alone line 3-3′ in FIG. 1.

FIG. 4 depicts a schematic view of an airway device in accordance with some embodiments of the present disclosure.

FIG. 5 depicts an airway device accordance with some embodiments of the present disclosure positioned in a patient's mouth.

To facilitate understanding, identical reference numerals have been used, where possible, to designate identical elements that are common to the figures. The figures are not drawn to scale and may be simplified for clarity. It is contemplated that elements and features of one embodiment may be beneficially incorporated in other embodiments without further recitation.

### DETAILED DESCRIPTION

Embodiments of the present invention provide an improved airway device that provides reduced trauma and complications during medical procedures as compared to conventional airway devices. Embodiments of the present disclosure may advantageously avoid or reduce morbidity by providing an airway configured to avoid the issues associated with conventional airway devices.

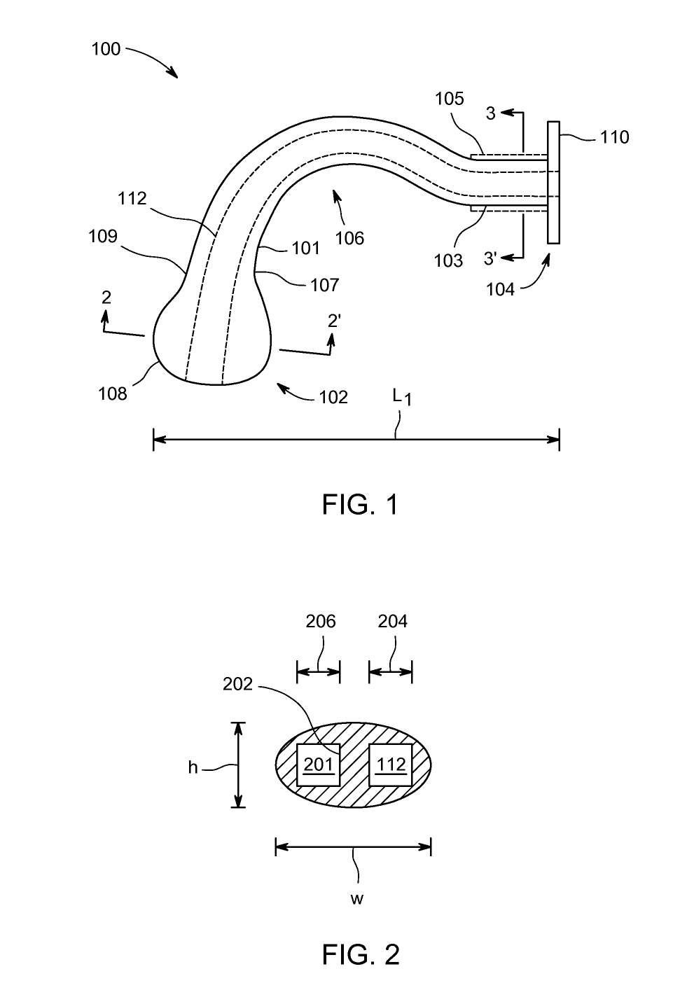

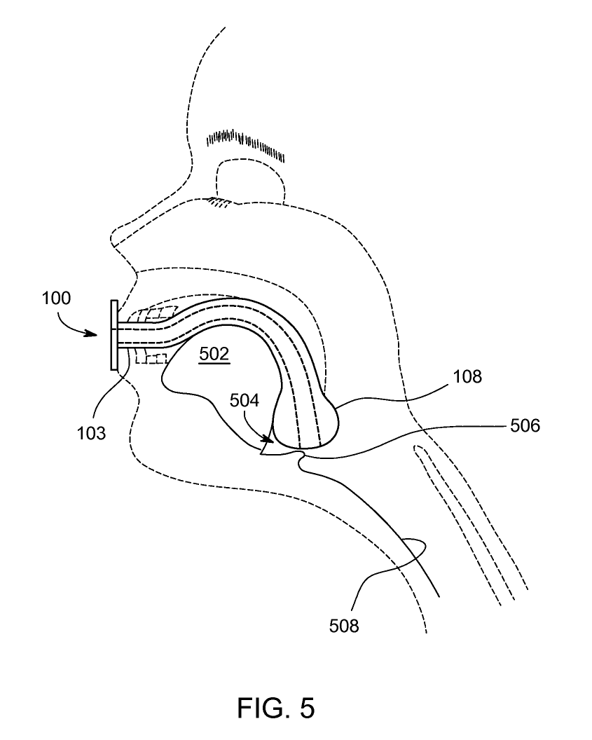

FIG. 1 depicts an airway device 100 in accordance with some embodiments of the present invention. In the following description, the terms distal and proximal are used with respect to a patient into whose mouth the airway device 100 is to be inserted. The airway device 100 includes a curved body 101 having a proximal end 102, a distal end, and a curved intermediate section 106 extending between the proximal and distal ends 102, 104. As will be discussed below, the curved body 101 is configured to sit on a patient's tongue. In some embodiments, an inside angle of curvature between the proximal and distal ends 102, 104 of the airway device 100 may be between 90° and 135°. The proximal end 102 includes a bulbous portion 108 configured to sit in the vallecular space of a patient's airway and provide an unobstructed path from the distal end 104 of the airway device to the patient's trachea. The distal end 104 includes a flange 110. During use, the bulbous portion 108 sits in the vallecular space of the patient's airway and the flange 110 is disposed outside of the patients mouth to prevent the airway device 100 from moving further down into the patient's airway, as discussed in further detail below with respect to FIG. 5.

The airway device 100 further includes at least one lumen 112 extending through the entire curved body 101 from the flange 110 to the bulbous portion 108. The airway device 100 has an overall length L₁ as shown in FIG. 1. Because a different sized airway device 100 is selected based on a size of the patient, the overall length L₁ varies depending on which airway device 100 size is selected. For example, the overall length L₁ may be between about 30 mm and about 125 mm.

In some embodiments, the airway device 100 may be formed of a pressure molded plastic for its rigidity, low mass, and partial flexibility. For example, the airway device may be formed of thermoplastic polyethylene polymers. In some embodiments, the airways device 100 may alternatively be formed more pliable material such as, for example, polyvinyl chloride (PVC). In such an embodiment, the at least one lumen 112 may be formed of a harder plastic such as a thermoplastic polyethylene polymer to ensure that the at least one lumen 112 retains patency during any flexing. The flange 110 would be contiguous with the at least one lumen 112, over which the PVC body would be disposed. Such a composition allows the remainder of the airway device 100 to be compliant enough to conform to the overall contour of a patient's airway from the lips to the vallecular space if the patient's head and neck are in a non-neutral position.

FIG. 2 depicts a cross sectional view through a maximum thickness of the bulbous portion 108. In some embodiments, the airway device 100 may include two lumens 112, 201 having respective widths 204, 206 and being separated by a partition 202, as illustrated in FIG. 2. In such an embodiment, one lumen 112 may be used as an air passageway and the other lumen 201 may be used to pass any type of medical tubing (e.g., a suction tube) therethrough. The partition 202 also advantageously serves to distribute compressive forces caused by a patient biting down on the airway device 100 and thus potentially blocking or partially obstructing the air passageway. In some embodiments, a thickness of the partition 202 between the lumens 112, 201 may be 1 mm. In some embodiments the thickness of the partition 202 between the lumens 112, 201 may be 2 mm. Because the partition 202 serves to buttress the airway device 100, the lumens 112, 201 may be sized so that the outer edges of the lumens 112, 201 are 1 mm or more away from an edge of the airway device 100.

At its maximum thickness, the bulbous portion 108 has a height h and a width w. As explained earlier, the size of the airway device 100 depends on the size of the patient being treated. In the example in which the overall length L₁ is 100 mm, the height h and the width w are 21 mm and 27 mm, respectively, and the widths 204, 206 are both 9 mm each. Although the lumens 112, 201 are depicted as having the same size, in some embodiments, the lumens 112, 201 may have different sizes. For example, when one lumen is larger than the other lumen, the smaller lumen may be used to pass a suction tube into the patient's airway and the larger lumen may be used as an airway. Regardless of whether the lumens 112, 201 have the same or different sizes, the sizes of the lumens 112, 201 are sized proportionally to the overall length L₁ of the airway device 100.

The bulbous portion 108 increases in size as the overall length L₁ of the airway device 100 increases such that the height h is increased more than the width w, which need only be slightly larger than the width of the remainder of the curved body 101. For example, a device with an overall length L₁ of 105 mm is sized such that the intermediate section 106 has a width of 20 mm and a height of 12 mm. The bulbous portion 108 of a 105 mm airway device 100 has a width w of 22 mm and a height h of 18 mm. As such, the width of the 105 mm airway device 100 increases by 10% from the intermediate section 106 to the bulbous portion, whereas the height increases by 50%. The length of the bulbous portion 108 is disposed within about 10° to about 20° of rotation as measured from the proximal end 102.

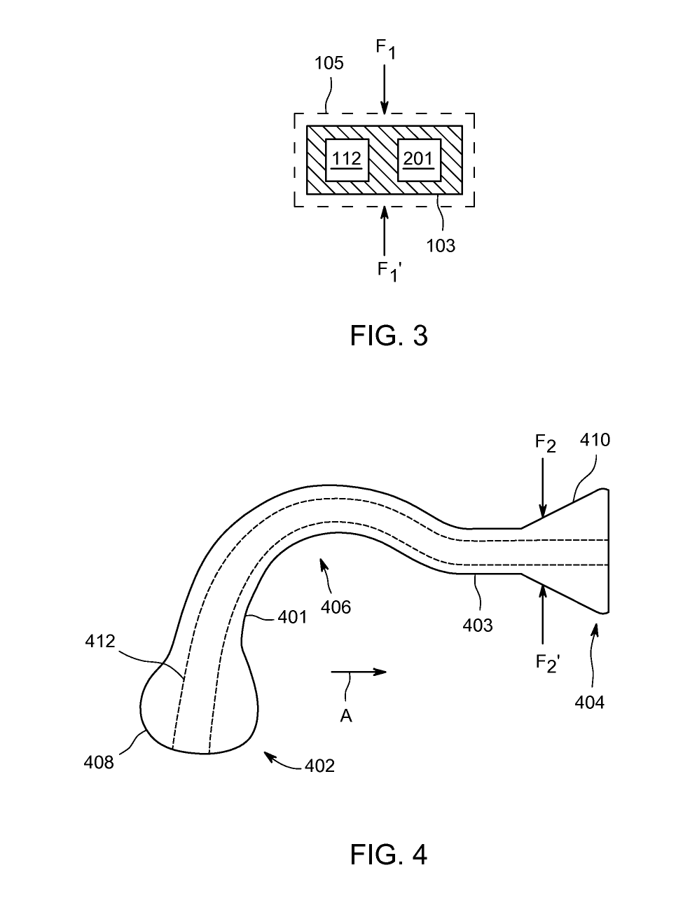

The inventor has discovered that a round or oval shaped cross-section results in the focusing of the compressive forces at one point on an airway device, thus resulting in trauma (e.g., damage to the patient's tooth/teeth). As such, the straight portion 103, which is disposed between the teeth of the patient during use, has a rectangular cross section, as will be discussed below with respect to FIG. 3. In some embodiments, the entirety of the curved body 101 except for the bulbous portion 108 has a rectangular cross section. An intermediate section 106 with a curved cross section may have a cross section with a height to width ratio between about 1:1 (i.e., square) to about 1:4 (i.e., rectangular). The dimensions of the cross section remain constant through the straight portion 103 and the intermediate section 106. In some embodiments, the straight portion 103 has a rectangular cross section while the intermediate section 106 alternatively has a curved cross section. Although the curved body 101 is shown as rectangular with sharp corners, in some embodiments, the curved body 101 may alternatively include rounded or beveled edges to further reduce any possible trauma to the patient caused by sharp edges.

Returning to FIG. 1, the airway device 100 includes a straight portion 103 disposed proximate the distal end 104. In the embodiment shown in FIG. 1, the straight portion is perpendicular to the flange 110. FIG. 3 depicts a cross-section taken through the straight portion 103. When a patient bites down on the straight portion 103, compressive forces (indicated in FIG. 3 by arrows F₁ and F₁′) are applied to the airway device 100. In addition to the partition 202, the rectangular shape of the straight portion 103 advantageously distributes the compressive forces throughout the curved body 101. In some embodiments, a jacket 105 (shown in phantom in FIGS. 1 and 3) may be disposed on the straight portion 103 to further alleviate any possible trauma to the patient when biting down on the airway device 100. The jacket 105 may be formed of an elastomeric material that is formed over the straight portion 103. For example, in some embodiments the jacket 105 may be formed of silicon.

The length of the straight portion 103 is such that it is disposed between the alveolar ridges (with or without teeth) during use to properly prevent occlusion of the mandibular and maxillary alveolar surfaces and enable the proper positioning of the bulbous portion 108 in the vallecular space. This ensures that the tongue and soft tissue of the oropharynx are pushed away to adequately define a conduit from the patient's mouth to the larynx. The straight portion 103 has a length sufficient to project into the patient's mouth, make contact with the surface of the tongue, and enable the airway device 100 to curve towards the back of the patient's head to follow the contour of the tongue. When the airway device 100 is used in smaller patients, for example, the length of the straight portion 103 may be as short as about 5 mm. When the airway device 100 is used in adults, the length of the straight portion 103 may be up to about 20 mm. In some embodiments, the straight portion 103 may have the same cross-sectional height and width as the intermediate section 106. A ratio of the cross-sectional height to width of the straight portion 103 may be between about 1:1 for a smaller airway device 100 (i.e., square) to about 1:4 for a larger airway device 100 (i.e., rectangular).

FIG. 4 depicts an airway device 400 in accordance with some embodiments of the present invention. The airway device 400 is substantially similar to the airway device 100 except that the distal end includes a tapered section 410 instead of a flange. Similar to the airway device 100, the airway device 400 includes a proximal end 402, a distal end 404, and an intermediate section 406 extending between the proximal and distal ends 402, 404. The proximal end 402 includes a bulbous portion 408. The tapered section 410 tapers from the distal end 404 towards the proximal end 402. A straight portion 403 extends from the proximal end of the tapered section 410.

The inventor has discovered that when a patient bites down, thus applying forces F₂ and F₂′, on the tapered section 410, the airway device moves in the direction indicated by arrow A (i.e., further out of the patient's mouth). As will be described in more detail below, with respect to FIG. 5, by moving in the direction indicated by arrow A, the airway device 400 further moves a patient's tongue and connected anatomy further out of the way of the patient's airway. The bulbous portion 408 and the straight portion 403 both have cross-sectional areas that are the same as those shown in FIGS. 2 and 3, respectively. Although not shown in FIG. 4, the jacket 105, described above with respect to FIGS. 1 and 3, may be fitted onto the straight portion 403.

FIG. 5 depicts the airway device 100 positioned within a patient's mouth. As shown in FIG. 5, the curved body 101 of airway device 100 sits on the patient's tongue 502 with the bulbous portion 108 resting in the vallecular space 504. The patient's epiglottis 506 is moved downward by the downward force exerted by the bulbous portion 108 on the frenulum connecting the epiglottis 506 and the tongue 502, thus providing an unobstructed path from the distal end 104 of the airway device 100 to the patient's trachea 508.

As explained above, trauma results from edges of conventional devices contacting mucosal surfaces of the patient's airway. The bulbous portion 108 advantageously avoids such trauma by eliminating any edge contact. The bulbous portion 108 further mitigates the previously described problems associated with poor sizing by filling the physiologic space in the vallecular space 504 of the SGA that results from the curvature of the tongue 502. The contour of the bulbous portion 108 enables the airway device 100 to dilate the retroglossal site of obstruction without moving from the desired position (shown in FIG. 5). Although the bulbous portion 108 is shown as sitting on the epiglottis 506, in some instances, the epiglottis 506 may alternatively be entirely pushed out of the way. The airway device 100 is shaped to advantageously avoid pressing on the base of the tongue 502, which causes a gag reflex.

Thus, improved airway devices for improving placement of the airway device and avoiding trauma to the patient have been disclosed herein. The contour and shape of the inventive airway device may advantageously allow for ease of placement of the airway device in a patient's mouth while eliminating or substantially reducing trauma caused to the patient.

While the foregoing is directed to embodiments of the present disclosure, other and further embodiments of the disclosure may be devised without departing from the basic scope thereof.

1. An airway device, comprising:
a curved body having a proximal end, a distal end, and an intermediate section extending between the proximal and distal ends;
at least one lumen extending through the curved body from the proximal end to the distal end;
a bulbous portion disposed at the proximal end; and
a straight portion disposed proximate the distal end between the distal end and the intermediate section.

2. The airway device of claim 1, further comprising:
a flange disposed at the distal end, wherein the straight portion is perpendicular to the flange.

3. The airway device of claim 1, further comprising:
a tapered section disposed at the distal end, wherein the tapered section tapers away from the distal end towards the proximal end, and wherein the straight portion extends from the tapered section towards the distal end.

4. The airway device of claim 1, further comprising:
a silicon jacket disposed over the straight portion.

5. The airway device of claim 1, wherein the at least one lumen includes two lumens.

6. The airway device of claim 1, wherein a curvature of the curved body between the proximal and distal ends is between about 90° to about 135°.

7. An airway device, comprising:
a curved body having a proximal end, a distal end, and an intermediate section extending between the proximal and distal ends;
at least one lumen extending through the curved body from the proximal end to the distal end;
a bulbous portion disposed at the proximal end;
a flange disposed at the distal end; and
a straight portion disposed proximate the distal end perpendicular to the flange.

8. The airway device of claim 7, further comprising:
a silicon jacket disposed over the straight portion.

9. The airway device of claim 7, wherein the at least one lumen includes two lumens.

10. The airway device of claim 7, wherein a curvature of the curved body between the proximal and distal ends is between about 90° to about 135°.

11. An airway device, comprising:
a curved body having a proximal end, a distal end, and an intermediate section extending between the proximal and distal ends;
at least one lumen extending through the curved body from the proximal end to the distal end;
a bulbous portion disposed at the proximal end;
a tapered section disposed at the distal end, wherein the tapered section tapers away from the distal end towards the proximal end; and
a straight portion disposed proximate the distal end extending from the tapered section towards the distal end.

12. The airway device of claim 11, further comprising:
a silicon jacket disposed over the straight portion.

13. The airway device of claim 11, wherein the at least one lumen includes two lumens.

14. The airway device of claim 11, wherein a curvature of the curved body between the proximal and distal ends is between about 90° to about 135°.
